(19) United States
(12) Patent Application Publication
(10) Pub. No.: US 2021/0056267 A1
(43) Pub. Date: Feb. 25, 2021

(54) METHOD FOR DETERMINING A REPRESENTATION OF A SUBJECTIVE STATE OF AN INDIVIDUAL WITH VECTORIAL SEMANTIC APPROACH

(71) Applicants: Oscar KJELL, MALMÖ (SE); Sverker SIKSTRÖM, LUND (SE); Katarina KJELL, MALMÖ (SE)

(72) Inventors: Oscar KJELL, MALMÖ (SE); Sverker SIKSTRÖM, LUND (SE); Katarina KJELL, MALMÖ (SE)

(21) Appl. No.: 17/040,697

(22) PCT Filed: Mar. 25, 2019
(86) PCT No.: PCT/SE2019/050264

§ 371 Date: Sep. 23, 2020

(30) Foreign Application Priority Data
Mar. 23, 2018 (SE) 1830097-0

(51) Int. Cl. G06F 40/30; G06N 3/08; G16H 50/20; G16H 10/20

(52) U.S. Cl. CPC G06F 40/30; G16H 10/20; G16H 50/20; G06N 3/08

(57) ABSTRACT

The disclosure relates to a method for determining a representation of a subjective state of an individual. The method comprises presenting a semantic question to the individual, and receiving at least one descriptive word obtained from the individual answering the semantic question. The method further comprises transforming each of said at least one descriptive word into a respective semantic representation vector thereby providing a set of one or more semantic representation vectors, and transforming the one or more semantic representation vectors in said set into one semantic representation vector. The method further comprises computing a first difference value or similarity value by comparing said one semantic representation vector with a first predetermined semantic representation of a state associated with a first mental illness or psychiatric disorder.

### FIELD OF INVENTION

The invention relates to a method for determining a representation of a subjective state of an individual. The invention also relates to a use of the method to provide a diagnosis of a first mental illness or psychiatric disorder. The invention also relates to a use of the method to provide a diagnosis distinguishing between a first and a second mental illness or psychiatric disorder.

The invention also relates to a computer program product on a non-transitory computer readable medium which computer program product when executed on a computer performs the method.

The invention also relates to a system for determining a representation of a subjective state of an individual.

### TECHNICAL BACKGROUND

In the field of psychology, psychological constructs such as emotions, thoughts and attitudes, are often measured by asking individuals to reply to questions using closed-ended numerical rating scales. Such rating scales are the dominant method used by professionals for assessing a patient's mental state. However, when asking people about their state of mind in a natural context (“How are you?”), we receive open-ended answers using words (“fine and happy!”) and not closed-ended answers using numbers (“7”). Thus, using rating scales require the patients to perform the cognitive task of translating their mental state into the one-dimensional response format to make it fit the scale.

Therefore, there exists a need for objectively quantifying responses without forcing the patient to perform the cognitive task of translating their mental state into a one-dimensional response to make it fit the rating scale.

### SUMMARY OF INVENTION

It is an object of the invention to alleviate at least some of the problems related to the prior art when trying to determine a representation of an individual's state of mind.

This object has been achieved by a method for determining a representation of a subjective state of an individual, the method comprising:

presenting a semantic question to the individual, receiving at least one descriptive word obtained from the individual answering the semantic question, transforming each of said at least one descriptive word into a respective semantic representation vector thereby providing a set of one or more semantic representation vectors, transforming the one or more semantic representation vectors in said set into one semantic representation vector,

computing a first difference value or similarity value by comparing said one semantic representation vector with a first predetermined semantic representation of a state associated with a first mental illness or psychiatric disorder.

This method has the advantage of taking an individual's open-ended response (i.e. the semantic answers) into account, as opposed to the prior art when only closed-ended answers (e.g. numbers on a rating scale) are considered and evaluated. The use of closed-ended answers can in some cases be inadequate or inaccurate when trying to understand an individual's state of mind, as their options for expressing themselves are limited. For example, patients suffering from depression and anxiety tend to give similar answers when responding to questions using closed-ended rating scales, but their responses differ more significantly when responding using open-ended answers.

Receiving at least one descriptive word obtained from the individual answering a semantic question typically involves the actions presenting a semantic question to the individual and receiving at least one descriptive word obtained from the individual answering the semantic question.

Moreover, the semantic representations from the word responses may be used in multiple regressions. This may be part of a training process or development process. It may be performed continuously or intermittently. It may be used to predict rating scale scores. It may also or alternatively be used to predict other outcomes such as categorising/classifying diagnoses.

The method may further comprise computing a second difference value or similarity value by comparing said one semantic representation vector with a second predetermined semantic representation of a state associated with a second mental illness or psychiatric disorder and thereby provide a set of data representing a similarity between the individual's state and the first mental illness or psychiatric disorder and a similarity between the individual's state and the second mental illness or psychiatric disorder. As stated above, this is particularly useful when wanting to substantiate between multiple mental illnesses or psychiatric disorders that are rated similar when using rating scales, but differ significantly when described semantically.

It may be noted that the use of term both difference value and similarity value is intended to relate to a value provided by the comparison between the semantic representation vectors. The value may be expressed as a difference or may be expressed as a similarity. The difference may be represented by a difference vector. The cosine of the angle may be computed to represent the semantic similarity between two semantic representations.

The method may further comprise comparing the first difference value or similarity value with the second difference value or similarity value to thereby provide a representation of a likelihood that the individual's subjective state is related to the first mental illness or psychiatric disorder compared to a likelihood that the individual's subjective state is related to the second mental illness or psychiatric disorder.

The method may further comprise that before presenting a semantic question to the individual, selects from a database containing a plurality of semantic questions a semantic question to be presented to an individual.

In accordance with the method the first mental illness or psychiatric disorder may be anxiety and the second mental illness or psychiatric disorder may be depression.

In accordance with the method, the predetermined semantic representation of a state associated with a mental illness or psychiatric disorder may be based on a semantic representation from a plurality of individuals' responses to one or more semantic questions related to characteristics of that mental illness or psychiatric disorder, optionally in combination with a self-assessment on a rating scale, and/or a clinically confirmed diagnosis of said mental illness or psychiatric disorder.

In accordance with the method, the one or more semantic representation vectors in said set may be transformed into one semantic representation vector by calculating the vector sum.

In accordance with the method, the respective semantic representation may be obtained via deep learning.

The present inventive concept further comprises a use of a method as disclosed above for determining a representation of a subjective state of an individual, to provide a diagnosis of a first psychiatric disorder.

The present inventive concept further comprises a use of a method as disclosed above for determining a representation of a subjective state of an individual, to provide a diagnosis distinguishing between a first and a second psychiatric disorder.

The present inventive concept further comprises a computer program product on a non-transitory computer readable medium which when executed on a computer performs a method for determining a representation of a subjective state of an individual.

The present inventive concept further comprises a system for determining a representation of a subjective state of an individual, the system comprising a control circuit configured to execute:

an input function configured to receive at least one descriptive word obtained from the individual answering a semantic question,

a first transformation function configured to transform each of said at least one descriptive word into a respective semantic representation vector thereby providing a set of one or more semantic representation vectors,

a second transformation function configured to transform the one or more semantic representation vectors in said set into one semantic representation vector, and

a first comparison function configured to compute a first difference value or similarity value by comparing said one semantic representation vector with a first predetermined semantic representation of a state associated with a first mental illness or psychiatric disorder.

In accordance with the system described above the control circuit is further configured to execute a second comparison function configured to compute a second difference value or similarity value by comparing said one semantic representation vector with a second predetermined semantic representation of a state associated with a second mental illness or psychiatric disorder and thereby a set of data representing a similarity between the individual's state and the first mental illness or psychiatric disorder and a similarity between the individual's state and the second mental illness or psychiatric disorder is provided.

It may be noted that the first and second comparison functions may be separate functions. In an alternative, the first and second comparison functions are the same function executed twice using the first predetermined semantic representation of a state associated with a first mental illness or psychiatric disorder as part of the data in the first execution and using the second predetermined semantic representation of a state associated with a second mental illness or psychiatric disorder as part of the data in the second execution.

In accordance with the system described above the control circuit may further be configured to execute

a likelihood determination function configured to compare the first difference value or similarity value with the second difference value or similarity value to thereby provide a representation of a likelihood that the individual's subjective state is related to the first mental illness or psychiatric disorder compared to a likelihood that the individual's subjective state is related to the second mental illness or psychiatric disorder.

In accordance with the system described above the control circuit may further be configured to, before the execution of the input function, execute:

a selection function configured to select from a database containing a plurality of semantic questions a semantic question to be presented to an individual, and

a presentation function configured to present the selected semantic question to the individual.

The method may also as a complement or as an alternative to the disclosure above be expressed in accordance with the following. The method comprises:

1. Creating a semantic representation for one mental states by aggregating a number of semantic representation associated with this mental state (e.g. anxiety) and normalizing this vector to the length of one.

2. Using the same procedure to create a semantic representation of the mental state.

3. Creating a difference vector, by taking the difference between the two vectors in 1 and 2, and then normalize the length of the vector to one.

4. Creating a semantic scale by measuring the semantic similarity between the difference vector in 3 and all semantic vectors, so that each mental is associated with one variable.

5. Repeating step 1 to 4 using a one-leave-out procedure so that difference vector always is generated without a different set of vectors compared to the one-vector that one compare with in step 4.

6. Calculate the t-test to compare values on one mental state with another mental state.

### BRIEF DESCRIPTION OF THE DRAWINGS

The invention will by way of example be described in more detail with reference to the appended schematic drawings, which shows a presently preferred embodiment of the invention.

FIG. 1 discloses an operational flowchart illustrating the steps carried out in a method for determining a representation of a subjective state of an individual according to the invention.

FIG. 2 discloses a system for determining a representation of a subjective state of an individual according to the invention.

### DETAILED DESCRIPTION OF PREFERRED EMBODIMENTS

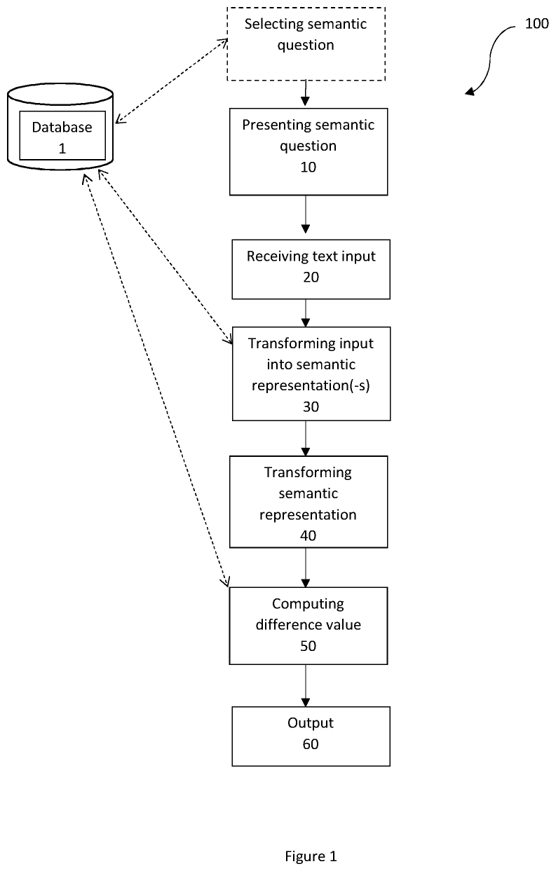

FIG. 1 discloses an operational flowchart illustrating the steps carried out in the method for determining a representation of a subjective state of an individual according to the invention. An individual is first presented (10) with an open-ended semantic question on a display. The individual answers the presented question with at least one descriptive word which is received (20) as text input. The question could be any question related to the individual's subjective state of mind, such as “How are you feeling?”. Each descriptive word is then transformed (30) into a respective semantic representation vector which provides a set of one or more semantic representation vectors. The one or more semantic representation vectors in the set are then transformed (40) into one semantic representation vector. The transformation (40) could for example comprise the one or more semantic representation vectors in the set to be transformed into one semantic representation vector by calculating the vector sum.

By comparing the one semantic representation vector with a first predetermined semantic representation of a state associated with a first mental illness or psychiatric disorder, a first difference value or similarity value can be computed (50).

By semantic representation a representation is meant in which natural language text, e.g. words, can be represented as vectors in a matrix called a semantic space. A semantic space is a high-dimensional matrix structure and the vectors representing natural language can be seen as coordinates in this space. The relation between two coordinates thus gives information about the similarity of the words', meaning. By representing an individual's open-ended answer as vectors in a high-dimensional space, the present inventive concept may complement and extend the one-dimensional response formats of current rating scales.

By predetermined semantic representation of a state associated with a first mental illness or psychiatric disorder, a coordinate or vector is meant that, based on previously obtained data, represents a certain mental illness or psychiatric disorder. The previously obtained data could be obtained in different ways, such as asking individuals with or without confirmed mental illnesses or psychiatric disorders to answer semantic questions in combination with rating scales. The respective semantic representations is optionally obtained via deep learning.

The term mental illness or psychiatric disorder is to be interpreted as depression or anxiety, or any of the following:

Acute stress disorder, Adjustment disorder, Adolescent antisocial behaviour, Adult antisocial behaviour, Agoraphobia, Alcohol abuse, Alcohol dependence, Alcohol withdrawal, Alcoholic hallucinosis, Alzheimer's disease, Amnestic disorder, Amphetamine dependence, Anorexia Nervosa, Anosognosia, Anterograde amnesia, Antisocial personality disorder, Anxiety disorder, Asperger syndrome, Atelophobia, Attention deficit disorder, Attention deficit hyperactivity disorder, Autism, Autophagia, Avoidant personality disorder, Avoidant/restrictive food intake disorder, Barbiturate dependence, Benzodiazepine dependence, Benzodiazepine misuse, Benzodiazepine withdrawal, Bereavement, Bibliomania, Binge eating disorder, Bipolar disorder, Bipolar I disorder, Bipolar II disorder, Body dysmorphic disorder, Borderline intellectual functioning, Borderline personality disorder, Brief psychotic disorder, Bulimia nervosa, Caffeine-induced anxiety disorder, Caffeine-induced sleep disorder, Cannabis dependence, Catatonia, Catatonic schizophrenia, Circadian rhythm sleep disorder, Claustrophobia, Cocaine dependence, Cocaine intoxication, Cognitive disorder, Communication disorder, Conduct disorder, Cotard delusion, Cyclothymia, Delirium tremens, Denial, Depersonalization disorder, Derealization, Dermatillomania, Desynchronosis, Developmental coordination disorder, Diogenes Syndrome, Dispareunia, Dissociative identity disorder, Dyscalculia, Dyspraxia, Dyslexia, EDNOS, Ekbom's Syndrome (Delusional Parasitosis), Encopresis, Epilepsy, Enuresis (not due to a general medical condition), Erotomania, Exhibitionism, Factitious disorder, Fregoli delusion, Fugue state, Furries, Ganser syndrome, Generalized anxiety disorder, General adaptation syndrome, Grandiose delusions, Gender identity disorder, Gaming disorder, Hallucinogen-related disorder, Hallucinogen persisting perception disorder, Histrionic personality disorder, Huntington's disease, Hypomanic episode, Hypochondriasis, Hysteria, Insomnia, Intermittent explosive disorder, Kleptomania, Korsakoff's syndrome Lacunar amnesia, Major depressive disorder, Major depressive episode, Male erectile disorder, Malingering, Manic episode, Mathematics disorder, Melancholia, Minor depressive disorder, Misophonia, Mixed episode, Mood disorder, Munchausen's syndrome, Narcissistic personality disorder, Narcolepsy, Neurocysticercosis, Neurodevelopmental disorder, Nicotine withdrawal, Night eating syndrome, Nightmare disorder, Obsessive-compulsive disorder (OCD), Obsessive-compulsive personality disorder (OCPD), Ondine's curse, Oneirophrenia, Opioid dependence, Opioid-related disorder, Oppositional defiant disorder (ODD), Orthorexia (ON), Pain disorder, Panic disorder, Paranoid personality disorder, Parasomnia, Parkinson's Disease, Partialism, Pathological gambling, Persecutory delusion, Personality disorder, Pervasive developmental disorder not otherwise specified (PDD-NOS), Phencyclidine (or phencyclidine-like)-related disorder, Phobic disorder, Pica (disorder), Psychosis, Phonological disorder, Physical abuse, Polysubstance-related disorder, Posttraumatic stress disorder (PTSD), Premature ejaculation, Primary hypersomnia, Primary insomnia, Pseudologia fantastica, Psychogenic amnesia, Psychotic disorder, Pyromania, Reactive attachment disorder of infancy or early childhood, Recurrent brief depression, Relational disorder, Residual schizophrenia, Retrograde amnesia, Rumination syndrome, Schizoaffective disorder, Schizoid personality disorder, Schizophrenia, Schizophreniform disorder, Schizotypal personality disorder, Seasonal affective disorder, Sedative-, hypnotic-, or anxiolytic-related disorder, Selective mutism, Separation anxiety disorder, Sexual fetishism, Sexual masochism disorder, Sexual sadism disorder, Shared psychotic disorder, Sleep disorder, Seasonal affective disorder, Sleep terror disorder, Sleepwalking disorder, Sleep paralysis, Social anxiety disorder, Social phobia, Somatization disorder, Somatoform disorder, Specific phobia, Stereotypic movement disorder, Stockholm syndrome, Stuttering, Substance-related disorder, Tardive dyskinesia, Transient global amnesia, Transient tic disorder, Transvestic disorder, and Trichotillomania. It may be noted that the term disorder does not necessarily need to relate to a state being accepted as diagnosis. It may be noted that that a specific subjective state of an individual may be considered differently in different countries. A specific subjective state of an individual may in some countries by considered a mental illness and in some countries be considered a diagnosed disorder whereas it in other countries it is not considered mental illness or a diagnosed disorder. Thus, the terms mental illness and psychiatric disorder as exemplified in this disclosure should be interpreted with such differences in mind. It may e.g. be noted that one such subjective state is fatigue syndrome which is a state which may not be considered as a disorder but which is a subjective state which may be said to relate to mental state or mental illness. Another such state is stress. As mentioned above, stress may e.g. be acute stress or posttraumatic stress. However, it may be noted that stress may also be a more diffuse state. It may e.g. be related to a long-term, low-impact disturbance or stress.

The computing step (50) may also comprise computing a second difference value or similarity value comparing said one semantic representation vector with a second predetermined semantic representation of a state associated with a second mental illness or psychiatric disorder. This second difference value or similarity value could thus provide a similarity between the individual's state and the first and second mental illnesses or psychiatric disorders.

If further, the computing step (50) compares the first difference value or similarity value with the second difference value or similarity value, a representation of a likelihood that the individual's subjective state is related to the first mental illness or psychiatric disorder compared to a likelihood that the individual's subjective state is related to the second mental illness or psychiatric disorder can be provided. This can especially be utilized when wanting to differentiate between similar disorders, such as anxiety and depression.

The method (100) may also comprise a database (1), in which a plurality of semantic questions is stored.

It is plausible that the method (100) can be used to provide a diagnosis of the first mental illness or psychiatric disorder. It could also be used for providing a diagnosis distinguishing between the first and the second mental illness or psychiatric disorder.

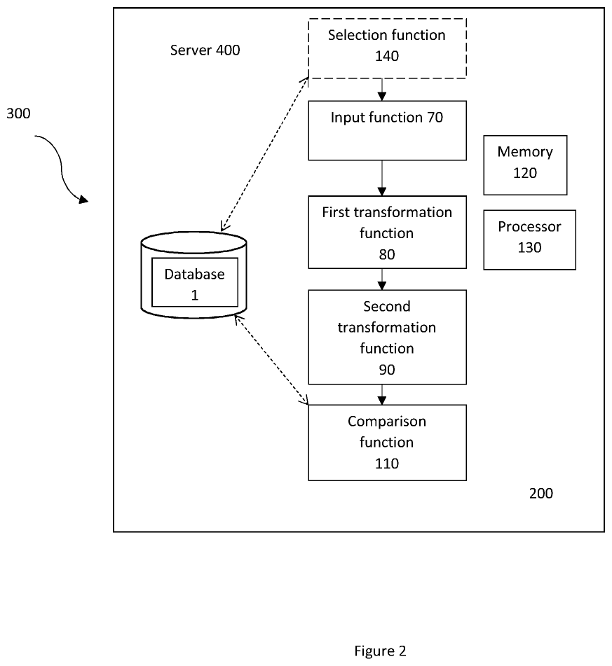

The system 300 described with reference to FIG. 2 for determining a representation of a subjective state of an individual comprises a control circuit 200. The control circuit 200 is configured to execute an input function 70 configured to receive at least one descriptive word obtained from the individual answering a semantic question. The control circuit 200 also executes a first transformation function 80 configured to transform each of said at least one descriptive word into a respective semantic representation vector thereby providing a set of one or more semantic representation vectors. The control circuit 200 is further configured to execute a second transformation function 90 configured to transform the one or more semantic representation vectors in said set into one semantic representation vector, and a first comparison function 110 configured to compute a first difference value or similarity value by comparing said one semantic representation vector with a first predetermined semantic representation of a state associated with a first mental illness or psychiatric disorder. The control circuit 200 is configured to carry out overall control of functions and operations of the central server 400 of the system 300. The control circuit 200 may include a processor 130, such as a CPU, microcontroller or microprocessor. The processor 130 is configured to execute program code stored in a memory 120, in order to carry out functions and operations of the control circuit 200. The memory 120 may be one or more of a buffer, a flash memory, a hard drive, a removable media, a volatile memory, a non-volatile memory, a random access memory (RAM), or another suitable device. In a typical arrangement, the memory may include a non-volatile memory for long term data storage and a volatile memory that functions as system memory for the control circuit 200. The memory 120 may exchange data with the control circuit 200 over a data bus. Accompanying control lines and an address bus between the memory 120 and the control circuit 200 also may be present. Functions and operations of the central server 400 may be embodied in the form of executable logic routines (e.g., lines of code, software programs, etc.) that are stored on a non-transitory computer readable medium (e.g., the memory 120) of the central server 400 and are executed by the control circuit 200 (e.g., using the processor 130). Furthermore, the functions and operations of the central server 400 may be a stand-alone software application or form a part of a software application that carries out additional tasks related to the central server 400. The described functions and operations may be considered a method that the corresponding device is configured to carry out. Also, while the described functions and operations may be implemented in software, such functionality may as well be carried out via dedicated hardware or firmware, or some combination of hardware, firmware and/or software.

The control circuit 200 can further be configured to execute a second comparison function 90 configured to compute a second difference value or similarity value by comparing said one semantic representation vector with a second predetermined semantic representation of a state associated with a second mental illness or psychiatric disorder and thereby a set of data representing a similarity between the individual's state and the first mental illness or psychiatric disorder and a similarity between the individual's state and the second mental illness or psychiatric disorder is provided.

The control circuit 200 can further be configured to execute a likelihood determination function configured to compare the first difference value or similarity value with the second difference value or similarity value to thereby provide a representation of a likelihood that the individual's subjective state is related to the first mental illness or psychiatric disorder compared to a likelihood that the individual's subjective state is related to the second mental illness or psychiatric disorder.

The control circuit 200 can further be configured to, before the execution of the input function 70, execute a selection function 140 configured to select from a database 1 containing a plurality of semantic questions a semantic question to be presented to an individual, and a presentation function configured to present the selected semantic question to the individual.

1. A method for determining a representation of a subjective state of an individual, the method comprising:
receiving at least one descriptive word obtained from the individual answering a semantic question;
transforming each of said at least one descriptive word into a respective semantic representation vector thereby providing a set of one or more semantic representation vectors;
transforming the one or more semantic representation vectors in said set into one semantic representation vector; and
computing a first difference value or a first similarity value by comparing said one semantic representation vector with a first predetermined semantic representation of a state associated with a first mental illness or psychiatric disorder.

2. The method according to claim 1, further comprising:
computing a second difference value or a second similarity value by comparing said one semantic representation vector with a second predetermined semantic representation of a state associated with a second mental illness or psychiatric disorder and thereby provide a set of data representing a similarity between the individual's state and the first mental illness or psychiatric disorder and a similarity between the individual's state and the second mental illness or psychiatric disorder.

3. The method according to claim 2, further comprising
comparing the first difference value or similarity value with the second difference value or similarity value to thereby provide a representation of a likelihood that the individual's subjective state is related to the first mental illness or psychiatric disorder compared to a likelihood that the individual's subjective state is related to the second mental illness or psychiatric disorder.

4. The method according to claim 1, further comprising, before receiving at least one descriptive word obtained from the individual answering a semantic question,
selecting from a database containing a plurality of semantic questions a semantic question to be presented to an individual, and
presenting the selected semantic question to the individual.

5. The method according to claim 2, wherein the first mental illness or psychiatric disorder is anxiety and the second mental illness or psychiatric disorder is depression.

6. The method according to claim 1, wherein the predetermined semantic representation of a state associated with a mental illness or psychiatric disorder is based on a semantic representation from a plurality of individuals' responses to one or more semantic questions related to characteristics of that mental illness or psychiatric disorder, optionally in combination with a self-assessment on a rating scale, and/or a clinically confirmed diagnosis of said mental illness or psychiatric disorder.

7. The method according to claim 1, wherein the one or more semantic representation vectors in said set is transformed into one semantic representation vector by calculating the vector sum.

8. The method according to claim 1, wherein the respective semantic representation is obtained via deep learning.

9. (canceled)

10. (canceled)

11. A computer program product on a non-transitory computer readable medium which computer program product when executed on a computer performs the actions comprising:
receiving at least one descriptive word obtained from the individual answering a semantic question;
transforming each of said at least one descriptive word into a respective semantic representation vector thereby providing a set of one or more semantic representation vectors;
transforming the one or more semantic representation vectors in said set into one semantic representation vector; and
computing a first difference value or a first similarity value by comparing said one semantic representation vector with a first predetermined semantic representation of a state associated with a first mental illness or psychiatric disorder.

12. A system for determining a representation of a subjective state of an individual, the system comprising:
a control circuit configured to execute:
an input function configured to receive at least one descriptive word obtained from the individual answering a semantic question;
a first transformation function configured to transform each of said at least one descriptive word into a respective semantic representation vector thereby providing a set of one or more semantic representation vectors;
a second transformation function configured to transform the one or more semantic representation vectors in said set into one semantic representation vector; and
a first comparison function configured to compute a first difference value or similarity value by comparing said one semantic representation vector with a first predetermined semantic representation of a state associated with a first mental illness or psychiatric disorder.

13. The system according to claim 12, wherein the control circuit is further configured to execute:
a second comparison function configured to compute a second difference value or similarity value by comparing said one semantic representation vector with a second predetermined semantic representation of a state associated with a second mental illness or psychiatric disorder and thereby a set of data representing a similarity between the individual's state and the first mental illness or psychiatric disorder and a similarity between the individual's state and the second mental illness or psychiatric disorder is provided.

14. The system according to claim 12, wherein the control circuit is further configured to execute:
a likelihood determination function configured to compare the first difference value or similarity value with the second difference value or similarity value to thereby provide a representation of a likelihood that the individual's subjective state is related to the first mental illness or psychiatric disorder compared to a likelihood that the individual's subjective state is related to the second mental illness or psychiatric disorder.

15. The system according to claim 12, wherein the control circuit is further configured to, before the execution of the input function, execute:
a selection function configured to select from a database containing a plurality of semantic questions a semantic question to be presented to an individual; and
a presentation function configured to present the selected semantic question to the individual.

16. The computer program product according to claim 11, further performing:
computing a second difference value or a second similarity value by comparing said one semantic representation vector with a second predetermined semantic representation of a state associated with a second mental illness or psychiatric disorder and thereby provide a set of data representing a similarity between the individual's state and the first mental illness or psychiatric disorder and a similarity between the individual's state and the second mental illness or psychiatric disorder.

17. The computer program product according to claim 16, further performing:
comparing the first difference value or similarity value with the second difference value or similarity value to thereby provide a representation of a likelihood that the individual's subjective state is related to the first mental illness or psychiatric disorder compared to a likelihood that the individual's subjective state is related to the second mental illness or psychiatric disorder.

18. The computer program product according to claim 11, further performing, before receiving at least one descriptive word obtained from the individual answering a semantic question,
selecting from a database containing a plurality of semantic questions a semantic question to be presented to an individual, and
presenting the selected semantic question to the individual.

19. The system according to claim 13, wherein the first mental illness or psychiatric disorder is anxiety and the second mental illness or psychiatric disorder is depression.

20. The system according to claim 12, wherein the predetermined semantic representation of a state associated with a mental illness or psychiatric disorder is based on a semantic representation from a plurality of individuals' responses to one or more semantic questions related to characteristics of that mental illness or psychiatric disorder, optionally in combination with a self-assessment on a rating scale, and/or a clinically confirmed diagnosis of said mental illness or psychiatric disorder.

21. The system according to claim 13, wherein the one or more semantic representation vectors in said set is transformed into one semantic representation vector by calculating the vector sum.

22. The system according to claim 12, wherein the respective semantic representation is obtained via deep learning.
